(19) United States
(12) Patent Application Publication
(10) Pub. No.: US 2026/0152463 A1
(43) Pub. Date: Jun. 4, 2026

(54) LOW-CARBON AND ENVIRONMENT-FRIENDLY METHOD FOR SYNTHESIZING PRIMARY AMINE FROM USING HALOGENATED HYDROCARBON

(71) Applicant: TAIZHOU CHUANGYUAN INDUSTRIAL TECHNOLOGY CO., LTD., Zhejiang (CN)

(72) Inventors: Changping XU, Zhejiang (CN); Yanrong JIA, Zhejiang (CN)

(73) Assignee: TAIZHOU CHUANGYUAN INDUSTRIAL TECHNOLOGY CO., LTD., Zhejiang (CN)

(21) Appl. No.: 19/464,568

(22) Filed: Jan. 29, 2026

Related U.S. Application Data
(63) Continuation of application No. PCT/CN2024/114717, filed on Aug. 27, 2024.

(30) Foreign Application Priority Data
Aug. 2, 2023 (CN) 202310964827.3

(51) Int. Cl. C07C 209/08; C07C 209/86

(52) U.S. Cl. CPC C07C 209/08; C07C 209/86

(57) ABSTRACT

A low-carbon and environment-friendly method for synthesizing a primary amine from a halogenated hydrocarbon in the technical field of organic synthesis is provided. The method includes adding formaldehyde and ammonia in an alcohol solution at a ratio of 8 mol:8 mol for a full reaction, then adding 3 mol of the halogenated hydrocarbon at a suitable temperature, followed by a full reaction, and performing a series of post-treatment steps to obtain a target primary amine product with relatively high purity.

### CROSS-REFERENCE TO RELATED APPLICATION

This application is a continuation of international application of PCT application serial no. PCT/CN2024/114717, filed on Aug. 27, 2024, which claims the priority benefit of China application serial no. 202310964827.3, filed on Aug. 2, 2023, now allowed. The entirety of each of the above-mentioned patent application is hereby incorporated by reference herein and made a part of this specification.

### TECHNICAL FIELD

This disclosure relates to the technical field of organic synthesis and, in particular, to a low-carbon and environment-friendly method for synthesizing a primary amine from a halogenated hydrocarbon.

### BACKGROUND

As is well known, there are numerous methods for synthesizing amine compounds, among which the most common methods using halogenated hydrocarbons as the primary raw material include: the Gabriel synthesis method, the Delepine synthesis method, and the method of direct reaction with a large excess of ammonia to obtain the target product.

Gabriel synthesis method: this method requires potassium phthalimide, hydrazine hydrate, polar solvents such as DMF, and similar substances as main raw materials. The market prices of these raw materials are relatively high, leading to elevated product costs. Furthermore, the process is relatively complex, and the separation and purification of the product are relatively cumbersome.

Delépine synthesis method: in this method, a halogenated hydrocarbon reacts with 1-1.1 equivalents of hexamethylenetetramine in a solvent such as chloroform to form a quaternary ammonium salt; after separating and removing the solvent, namely chloroform, the quaternary ammonium salt is hydrolyzed in methanol (or ethanol) in an amount 12 times or more that of the halogenated hydrocarbon, under the action of an acid in an amount 3 times or more that of the halogenated hydrocarbon, yielding an ammonium salt of the target product, an acetal in an amount 6 times or more that of the halogenated hydrocarbon, and an inorganic ammonium salt in an amount 3 times or more that of the halogenated hydrocarbon; and after separating the acetal, the target primary amine product is obtained by adjusting the pH with a base. Although the raw material cost of this method is lower than that of the Gabriel synthesis method, it generates a great amount of low-recovery-value acetal and inorganic ammonium salt during synthesis, leading to a waste of resources and environmental treatment pressure. Moreover, the initial reaction in chloroform to form the quaternary ammonium salt, followed by the subsequent separation of chloroform, increases the complexity of the operational steps.

The reaction scheme is as follows:

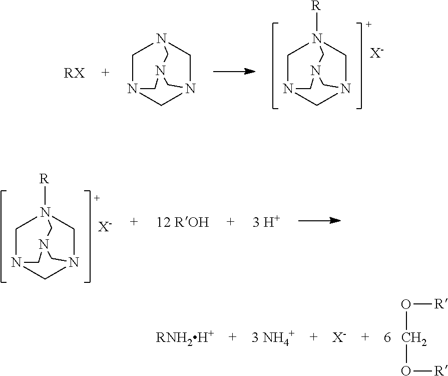

Method of direct reaction with excess ammonia: since the target primary amine product readily undergoes further reaction with the halogenated hydrocarbon as a raw material to form secondary amines, tertiary amines, and even quaternary ammonium salts, the ammonia used as a raw material must be in great excess. Nevertheless, even under such conditions, small amounts of by-products such as secondary and tertiary amines are generated, resulting in low purity of the target primary amine product and difficulties in separation. This method is suitable for the synthesis of amines, where primary, secondary, and tertiary amines all possess recovery value and can be mutually separated.

### SUMMARY

In view of the problems existing in the prior art, this disclosure aims to provide a low-carbon and environment-friendly method for synthesizing a primary amine from a halogenated hydrocarbon, specifically achieved through the following technical solution:
a low-carbon and environment-friendly method for synthesizing a primary amine from a halogenated hydrocarbon, including the following steps:
1) adding formaldehyde or paraformaldehyde and a sufficient amount of an alcohol solvent into a four-necked flask equipped with a stirrer, a thermometer, a reflux condenser, and a dropping funnel, cooling in an ice-water bath and adding aqueous ammonia dropwise under stirring to obtain a mixture, controlling a temperature of the mixture from 0° C. to a reflux temperature until the mixture is completely dissolved, and then continuing stirring for 0-2 h;
2) adding the halogenated hydrocarbon dropwise into the four-necked flask, stirring and heating in a water bath, and allowing for a full reaction at a temperature from 20° C. to a reflux temperature for 0.5-24 h;
3) after the reaction is complete, cooling slightly, controlling the temperature at 0-60° C., adding 0.9-1.5 A equivalents of an inorganic acid or an organic acid dropwise under stirring, where A refers to a molar amount of ammonia contained in the aqueous ammonia minus a molar amount of the halogenated hydrocarbon, adding a small amount of water if necessary, and allowing for a reaction under stirring at a temperature of 0-60° C. for 0.5-10 h; and
4) removing and replacing the reflux condenser with a distillation apparatus, adding an appropriate amount of water if necessary until all the organic solvents are distilled out, and then proceeding with a post-treatment to obtain a finished primary amine product.

Further, in the step 1), the temperature is controlled at preferably 15-30° C.; the alcohol solvent is preferably any one of methanol or ethanol; and the stirring continued upon dissolution lasts for preferably 20-60 min.

If the paraformaldehyde is fed, a molar amount of the paraformaldehyde should be converted into an equivalent molar amount of the formaldehyde.

Further, a molar feed ratio of the halogenated hydrocarbon, the formaldehyde, and the ammonia contained in the aqueous ammonia is 1.0:(1.0-10.0):(2.0-10.0), and the molar amount of the ammonia contained in the aqueous ammonia is ≥the molar amount of the formaldehyde; and
a feed amount of the alcohol should be sufficient to ensure that the reaction mixture is in a solution state after the dropwise addition of the halogenated hydrocarbon in the subsequent step 2), and an actual amount of the alcohol varies depending on the halogenated hydrocarbon, but should at least be greater than twice the molar amount of the formaldehyde, as the alcohol not only serves as a solvent but also reacts as a reactant with formaldehyde generated during hydrolysis upon subsequent addition of hydrochloric acid for the hydrolysis, forming acetal to remove the formaldehyde.

Further, the molar feed ratio of the halogenated hydrocarbon, the formaldehyde, and the ammonia contained in the aqueous ammonia is preferably 1.0:(2.6-3.2):(2.6-3.5), and the molar amount of the ammonia contained in the aqueous ammonia is ≥the molar amount of the formaldehyde.

Further, in the step 2), after the dropwise addition of the halogenated hydrocarbon, the heating in the water bath is performed at preferably 30-65° C.; and the full reaction lasts for preferably 1-12 h.

Further, in the step 3), the temperature is appropriately controlled at preferably 20-50° C.; the acid is added dropwise in an amount of preferably 1.0-1.3 A equivalents of acid, where A refers to the molar amount of the ammonia contained in the aqueous ammonia minus the molar amount of the halogenated hydrocarbon; the acid is preferably hydrochloric acid as the inorganic acid; and the reaction under stirring lasts for preferably 1-5 h.

The purpose of adding a small amount of water is to dissolve a small amount of substances that are not fully soluble, thereby forming a homogeneous solution. If all the substances can form the homogeneous solution upon the addition of the hydrochloric acid, then it is unnecessary to add water.

Further, in the step 4), the distillation apparatus employs atmospheric distillation or reduced-pressure distillation, where for some materials with poor thermal stability, the reduced-pressure distillation may be used to lower the temperature for protection; and
the appropriate amount of water means that the amount of water added is sufficient to dissolve ammonium chloride precipitated during a later stage of the distillation, so that all the organic solvents can be distilled out normally.

The solvents distilled out primarily include the alcohol and the acetal, along with a small amount of water. After separation, most of the alcohol can be recovered. In the industrial application of the technology disclosed herein, the recovered alcohol can be reused as a solvent for the next batch of reactions.

Further, in the step 4), the post-treatment employs the following different methods, specifically:
method A: slightly cooling and adjusting a distillation residue to strong alkalinity with an aqueous solution of an alkali metal or alkaline earth metal hydroxide in an amount of 0.9-1.5 B equivalents, standing for layer separation, extracting an aqueous layer with a suitable extractant and then transferring to an environment-friendly treatment system, combining an extract and an oil layer, removing water and performing fractional distillation under reduced pressure, and collecting an appropriate fraction to obtain the finished primary amine product; or
method B: cooling a distillation residue with ice water for crystallization, vacuum filtering until dry, adjusting obtained crystals to strong alkalinity with an aqueous solution of an alkali metal or alkaline earth metal hydroxide in an amount of 0.9-1.5 B equivalents, standing for layer separation, extracting an aqueous layer with a suitable extractant and then transferring to an environment-friendly treatment system, combining an extract and an oil layer, removing water and performing fractional distillation under reduced pressure, and collecting an appropriate fraction to obtain the finished primary amine product; or
method C: after adjusting a distillation residue to strong alkalinity by the method A or method B, performing steam distillation, allowing a distillate to stand for layer separation, extracting an aqueous layer with a suitable extractant and then transferring to an environment-friendly treatment system, combining an extract and an oil layer, removing water and performing fractional distillation under reduced pressure, and collecting an appropriate fraction to obtain the finished primary amine product;
where in the above methods, B refers to the molar amount of the acid added plus the molar amount of the halogenated hydrocarbon added; and the extractant is selected from an ether substance, a homologue of benzene, or some halogenated hydrocarbons.

Further, the aqueous solution of the alkali metal or alkaline earth metal hydroxide is preferably an aqueous sodium hydroxide solution or an aqueous potassium hydroxide solution in an amount of preferably 1.0-1.3 B, where B refers to the molar amount of the acid added plus the amount of the molar halogenated hydrocarbon added;
the ether substance is preferably any one of diethyl ether, propyl ether, isopropyl ether, butyl ether, methyl tert-butyl ether, or anisole; the homologue of benzene is preferably any one of benzene, toluene, or xylene; the some halogenated hydrocarbons are preferably dichloromethane;
a method for removing water is as follows: removing water by drying with an inorganic salt followed by filtration, where the inorganic salt is preferably any one of anhydrous potassium carbonate, anhydrous sodium carbonate, or anhydrous sodium sulfate;
or, utilizing a characteristic of the extractant capable of forming an azeotropic mixture with water, heating under reflux, and separating the water therein using a water separator.

The principle of this disclosure is as follows:
in the alcohol solvent, when the ratio of the formaldehyde (or paraformaldehyde, which must be converted into an equivalent amount of the formaldehyde) to the ammonia is 1 mol: 1 mol, the following reaction occurs to form a cyclic compound, namely 1,3,5-hexahydrotriazine, and the reaction is a reversible reaction.

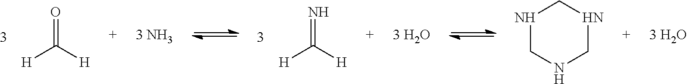

When the amount of the ammonia is insufficient, especially when the ratio of the formaldehyde to the ammonia is 6 mol:4 mol, the following reaction occurs to form hexamethylenetetramine, and the reaction is also a reversible reaction.

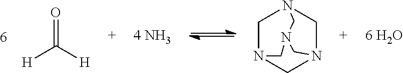

If initial feed amounts of both the formaldehyde and the ammonia are, for example, 8 mol, a maximum of 8/3 mol of the cyclic compound, namely 1,3,5-hexahydrotriazine, can form in the solution. At this point, if the halogenated hydrocarbon is added, the following reaction occurs:

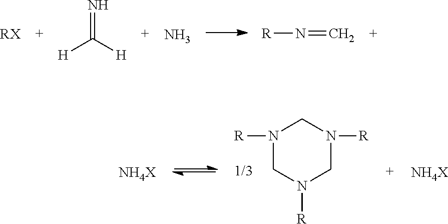

or is represented by the following reaction scheme:

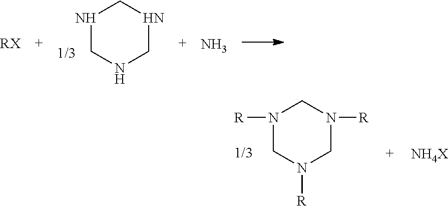

When 1 mol of halogenated hydrocarbon has reacted, 1/3 mol of 1,3,5-trialkylhexahydrotriazine can be generated, and 1 mol of ammonia is consumed to form 1 mol of inorganic ammonium halide. This changes the ratio of the formaldehyde to the ammonia in the reaction system to 7 mol: 6 mol. At this point, due to the insufficient amount of ammonia, there is a tendency to form the cage compound, namely hexamethylenetetramine, in the reaction system.

When 2 mol of halogenated hydrocarbon have reacted, 2/3 mol of 1,3,5-trialkylhexahydrotriazine can be generated, and 2 mol of ammonia are consumed to form 2 mol of inorganic ammonium halide. This changes the ratio of the formaldehyde to the ammonia in the reaction system to 6 mol: 4 mol, which precisely allows for the formation of 1 mol of hexamethylenetetramine. The 1 mol of hexamethylenetetramine can form a quaternary ammonium salt with 1 mol of halogenated hydrocarbon.

Therefore, after 8 mol of formaldehyde and 8 mol of ammonia are dissolved and reacted in an alcohol solution, by adding 3 mol of halogenated hydrocarbon and allowing for a full reaction, 2/3 mol of 1,3,5-trialkylhexahydrotriazine and 1 mol of the aforementioned N-alkylhexamethylenetetramine quaternary ammonium salt are formed.

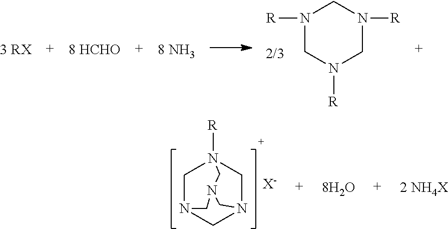

Upon hydrolysis under the action of the acid and the alcohol, 1 mol of the aforementioned quaternary ammonium salt is converted, according to the principle of the Delépine synthesis method, into 1 mol of the ammonium salt of the target product, 6 mol of acetal, and 3 mol of inorganic ammonium salt. Similarly, 2/3 mol of 1,3,5-trialkylhexahydrotriazine is converted into 2 mol of the ammonium salt of the target product and 2 mol of acetal, according to an analogous principle.

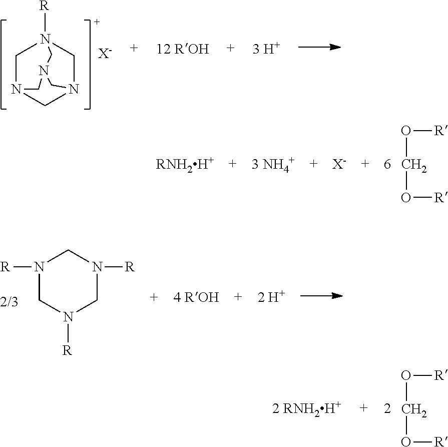

The resulting ammonium salt of the target product is subjected to distillation to separate and remove the acetal. Then, through various post-treatment steps and adjustment to strong alkalinity with an alkali solution, followed by separation and purification, the finished primary amine product is obtained.

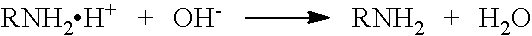

The overall reaction steps of this disclosure can be summarized as follows:
1. the reaction of 8 mol of formaldehyde with 8 mol of ammonia yields a maximum of 8/3 mol of 1,3,5-hexahydrotriazine. This is a reversible reaction, as shown in the following reaction scheme:

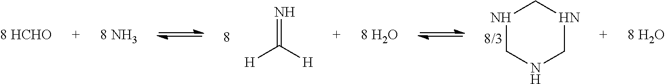

2. The reaction of 3 mol of halogenated hydrocarbon with 8/3 mol of 1,3,5-hexahydrotriazine yields 2/3 mol of 1,3,5-trialkylhexahydrotriazine and 1 mol of N-alkylhexamethylenetetramine quaternary ammonium salt, concurrently yielding 2 mol of inorganic ammonium halide as a by-product. The reaction scheme is as follows:

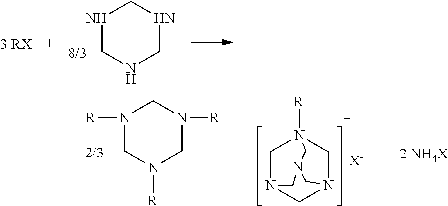

3. The hydrolysis of 2/3 mol of 1,3,5-trialkylhexahydrotriazine under the action of 4 mol of alcohol and 2 mol of acid yields 2 mol of the ammonium salt of the target product and 2 mol of acetal. The reaction scheme is as follows:

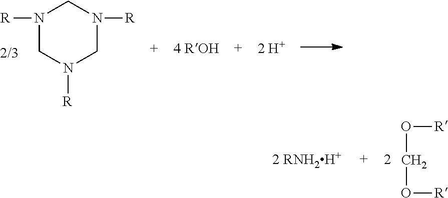

The hydrolysis of 1 mol of N-alkylhexamethylenetetramine quaternary ammonium salt under the action of 12 mol of alcohol and 3 mol of acid yields 1 mol of the ammonium salt of the target product, 6 mol of acetal, and 3 mol of inorganic ammonium salt. The reaction scheme is as follows:

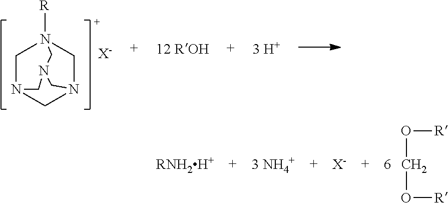

4. The ammonium salt of the target product is adjusted to strong alkalinity with an alkali solution, precipitating the target primary amine product. The reaction scheme is as follows:

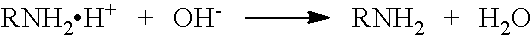

The above is a relatively intuitive and straightforward explanation employed by the inventor to illustrate the principle of this disclosure. The actual reaction pathway may be far more complex. Experimental results have confirmed that when the ratio of the halogenated hydrocarbon, the formaldehyde, and the ammonia contained in the aqueous ammonia is 3 mol: 8 mol: 8 mol, the yield of the target product is the highest, generally reaching over 80%. Further increasing the amounts of the formaldehyde and the ammonia proportionally does not significantly enhance the yield. For example, when the ratio of the halogenated hydrocarbon, the formaldehyde, and the ammonia contained in the aqueous ammonia is 3 mol: 12 mol: 12 mol, the yield of the primary amine shows no substantial change.

Given that the market prices of the formaldehyde and the aqueous ammonia are relatively inexpensive compared to the halogenated hydrocarbon, and the aqueous ammonia is prone to volatilization and escape, the amounts of the formaldehyde and the aqueous ammonia are slightly increased in the experiments, ensuring that the molar amount of the ammonia contained in the aqueous ammonia is ≥the molar amount of the formaldehyde.

When the feed amounts of the formaldehyde and the ammonia are reduced, the target product is still generated, but the yield decreases. For example: when the ratio of the halogenated hydrocarbon, the formaldehyde, and the ammonia contained in the aqueous ammonia is 1 mol: 1 mol: 2 mol, the experimental yield can still reach over 60%, though it is not as high as that generated when the ratio of the three is 3:8:8. When the amount of the formaldehyde is reduced, the amount of the ammonia can also be reduced, but the extent of ammonia reduction should be less than that of formaldehyde reduction. For example, when the ratio of the halogenated hydrocarbon and the formaldehyde changes from 3:8 to 1:1, the ratio of the halogenated hydrocarbon and the ammonia contained in the aqueous ammonia should change from 3:8 to 1:2.

This disclosure has further discovered that aryl methyl halides, allylic halogenated hydrocarbons, and certain halogenated compounds where the carbon atom attached to the halomethyl group bears multiple bonds are applicable to the method of this disclosure. However, halogenated aliphatic alkanes do not perform well. Experiments using tert-butylchloride and 1-bromooctane as main raw materials both failed to synthesize the corresponding primary amines.

Compared with the Delepine reaction, the technical solution of this disclosure primarily offers the following beneficial effects:
1) reduced raw material consumption, lower carbon footprint, and decreased raw material cost for the product.

In the Delepine reaction, theoretically, 1 mol of halogenated hydrocarbon reacts with 1 mol of hexamethylenetetramine to produce 1 mol of quaternary ammonium salt. Here, 1 mol of hexamethylenetetramine is equivalent to 6 mol of formaldehyde and 4 mol of ammonia. The obtained 1 mol of quaternary ammonium salt hydrolyzes under the action of 12 mol of alcohol and 3 mol of acid, yielding 1 mol of the ammonium salt of the target product, along with 6 mol of acetal and 3 mol of inorganic ammonium salt. After removing the acetal, separating the ammonium salt of the target product from the inorganic ammonium salt is often economically unfeasible. Consequently, both are typically adjusted directly to strong alkalinity with an alkali solution without separation, consuming over 4 mol of alkali. This results in the precipitation of, at most, 1 mol of the target primary amine product and 3 mol of inorganic ammonia.

In this disclosure, when the ratio of the halogenated hydrocarbon, the formaldehyde, and the ammonia contained in the aqueous ammonia is 3:8:8, theoretically, after 8/3 mol of formaldehyde and 8/3 mol of ammonia react, they continue to react with 1 mol of halogenated hydrocarbon, yielding 2/9 mol of 1,3,5-trialkylhexahydrotriazine and 1/3 mol of N-alkylhexamethylenetetramine quaternary ammonium salt. Hydrolysis of both under the action of 16/3 mol of alcohol and 5/3 mol of acid yields 1 mol of the ammonium salt of the target product, along with 8/3 mol of acetal and 5/3 mol of inorganic ammonium salt. After removing the acetal, separating the ammonium salt of the target product from the inorganic ammonium salt is often economically unfeasible. Consequently, both are typically adjusted directly to strong alkalinity with an alkali solution without separation, consuming over 8/3 mol of alkali. This results in the precipitation of, at most, 1 mol of the target primary amine product and 5/3 mol of inorganic ammonia.

Table 1 lists the theoretical minimum consumptions of raw materials required to yield 1 mol of the target primary amine product by the Delépine synthesis method and this disclosure.

TABLE 1

| | Raw material | | | | | | |
|---|---|---|---|---|---|---|---|
| | | | Halogenated | | | Hydrochloric | Sodium |
| | Formaldehyde | Ammonia | hydrocarbon | Chloroform | Alcohol | acid | hydroxide |
| Method | (mol) | (mol) | (mol) | (L) | (mol) | (mol) | (mol) |
| Delépine | 6 | 4 | 1 | Approximately | 12 | 3 | 4 |
| synthesis | | | | 1 required | | | |
| method | | | | | | | |
| Method of | 8/3 | 8/3 | 1 | 0 | 16/3 | 5/3 | 8/3 |
| this | | | | | | | |
| disclosure | | | | | | | |

Obviously, this disclosure requires less raw material consumption, has a lower carbon footprint, and results in lower raw material costs for the product.

(Note: since the methanol in this disclosure serves both as a solvent and as a reactant, its actual consumption per batch may appear significantly higher than the theoretical amount. However, in large-scale production applications, the recovered methanol, after separation and removal of impurities such as acetal, can be reused in the production of the next batch. This recycling and reuse will substantially reduce the actual consumption of methanol.)
2) Lower output of by-products including acetal and inorganic ammonia, and better environment-friendliness

From the above analysis, it is evident that in the Delépine reaction, theoretically, the production of 1 mol of the target product generates 6 mol of acetal and 3 mol of inorganic ammonia. In contrast, this disclosure generates only 8/3 mol of acetal and 5/3 mol of inorganic ammonia per 1 mol of the target product produced. Both the acetal and the inorganic ammonia are compounds with low recovery value, and their environmental treatment incurs high costs. Therefore, the synthetic process of this disclosure is greener and more environment-friendly.

Table 2 lists the theoretical amounts of by-products including acetal and ammonia generated per 1 mol of the target primary amine product produced by the Delépine synthesis method and this disclosure.

TABLE 2

By-product

| Method | Acetal (mol) | Ammonia (mol) |
|---|---|---|
| Delépine synthesis | 6 | 3 |
| method | | |
| Method of this | 8/3 | 5/3 |
| disclosure | | |

3) Simpler synthetic process

The Delépine reaction involves first synthesizing hexamethylenetetramine from formaldehyde and ammonia in a molar ratio of 6:4. Due to the poor solubility of hexamethylenetetramine in alcohol solvents but its good solubility in chloroform, the prepared hexamethylenetetramine can only be dissolved in chloroform before reacting with the halogenated hydrocarbon. After the reaction is complete, the chloroform must be thoroughly separated. Subsequently, hydrolysis into the target ammonium salt product occurs under the action of alcohol solvents such as methanol and an acid, resulting in a complex and cumbersome process.

In contrast, this disclosure directly dissolves and reacts formaldehyde and ammonia in a molar ratio of 1:1 in an alcohol solvent such as methanol. Then, the halogenated hydrocarbon is added for a full reaction. After the reaction is complete, the mixture is cooled to a suitable temperature, and an acid such as hydrochloric acid is added for hydrolysis into the target ammonium salt product. The entire reaction consistently takes place within the same reactor, resulting in a simple process with easy operation.
4) Product yield generally over 80%

When the feed ratio of the reactants, namely, the halogenated hydrocarbon, the formaldehyde, and the ammonia contained in the aqueous ammonia, is close to 3:8:8, the product yield of this disclosure is generally over 80%, with yields for some products reaching over 85%. This is merely a single-pass yield, established without recycling and reusing the raw materials such as the extractant or the head and tail fractions generated during fractional distillation under reduced pressure. If the raw materials like the extractant and the head and tail fractions generated during fractional distillation under reduced pressure are recovered and reused, the yield would be even higher, making it highly possible to exceed 90%.

In summary, this disclosure possesses a high degree of innovation, and the resulting beneficial effects are significant. In this disclosure, a portion of the halogenated hydrocarbon reacts with the formaldehyde and the ammonia to form the N-alkylhexamethylenetetramine quaternary ammonium salt. The reaction pathway proceeds according to the Delépine synthesis principle. Therefore, this disclosure can be regarded as an application and extension of the Delépine synthesis method.

### BRIEF DESCRIPTION OF THE DRAWINGS

FIG. 1 shows a gas chromatogram and content analysis of benzylamine as a product of Embodiment 1;

FIG. 2 shows a proton nuclear magnetic resonance (¹H NMR) spectrum of the benzylamine as the product of Embodiment 1;

FIG. 3 shows a gas chromatogram and content analysis of 2-chlorobenzylamine as a product of Embodiment 2;

FIG. 4 shows a ¹H NMR spectrum of the 2-chlorobenzylamine as the product of Embodiment 2;

FIG. 5 shows a gas chromatogram and content analysis of 4-fluorobenzylamine as a product of Embodiment 3;

FIG. 6 shows a ¹H NMR spectrum of the 4-fluorobenzylamine as the product of Embodiment 3;

FIG. 7 shows a gas chromatogram and content analysis of 4-tert-butylbenzylamine as a product of Embodiment 4;

FIG. 8 shows a ¹H NMR spectrum and an amplified aromatic region spectrum of the 4-tert-butylbenzylamine as the product of Embodiment 4;

FIG. 9 shows a gas chromatogram and content analysis of 2,4-dichlorobenzylamine as a product of Embodiment 5;

FIG. 10 shows a ¹H NMR spectrum of the 2,4-dichlorobenzylamine as the product of Embodiment 5;

FIG. 11 shows a gas chromatogram and content analysis of 4-bromobenzylamine as a product of Embodiment 6;

FIG. 12 shows a ¹H NMR spectrum of the 4-bromobenzylamine as the product of Embodiment 6;

FIG. 13 shows a gas chromatogram and content analysis of 1-naphthalenemethylamine as a product of Embodiment 7; and

FIG. 14 shows a ¹H NMR spectrum of the 1-naphthalenemethylamine as the product of Embodiment 7.

### DETAILED DESCRIPTION OF THE EMBODIMENTS

This disclosure will be further described below in conjunction with specific embodiments to facilitate a better understanding of the technical solution.

### Embodiment 1

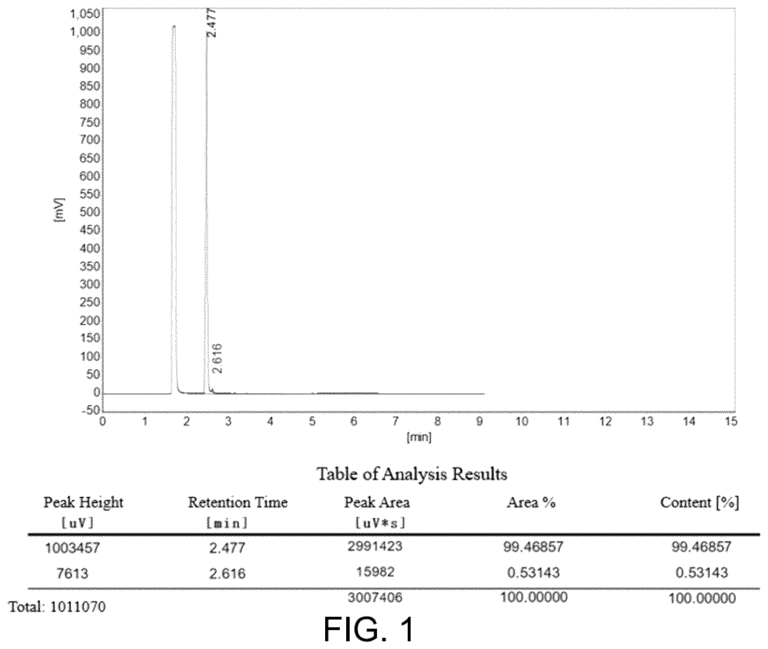

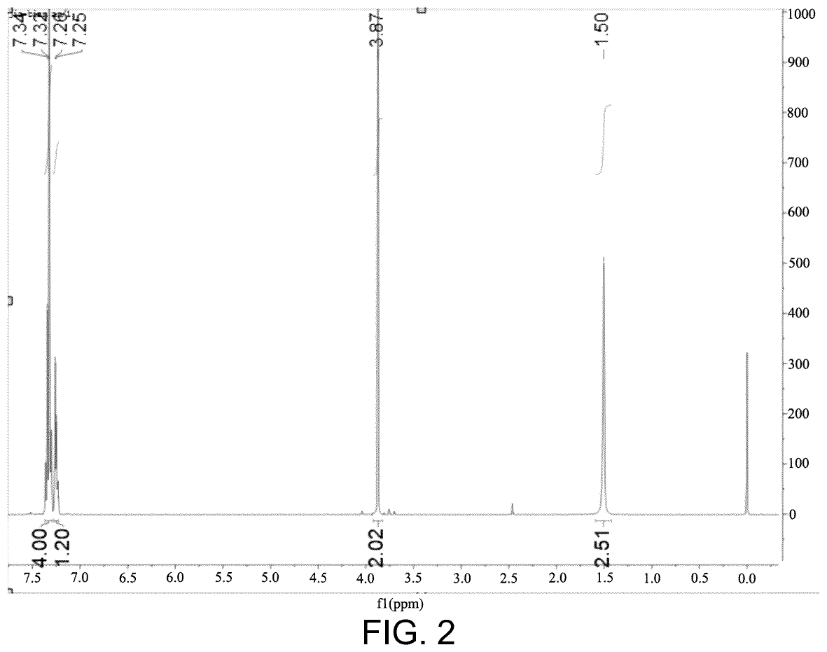

33.63 g of paraformaldehyde (1.12 mol) and 310 mL of methanol were added into a 500 mL four-necked flask equipped with a stirrer, a thermometer, a reflux condenser, and a dropping funnel to form a mixture; the mixture was cooled in an ice-water bath, and 79.77 g of aqueous ammonia with a mass concentration of 25.62% (1.2 mol) were added under stirring, with the temperature controlled below 30° C.; after the mixture was completely dissolved and clarified, stirring was continued for 20 min; then, 50.63 g of benzyl chloride (0.4 mol) were added; the water bath temperature was raised to control the reaction temperature at 58-60° C.; after stirring for 6 h, the reaction was stopped and cooled to room temperature with cold water; 92 g of 36.5% hydrochloric acid (0.92 mol) were added; the mixture was heated in a water bath, with the reaction temperature controlled at 44-46° C.; after reaction for 2 h, an atmospheric distillation apparatus was used to distill out all organic solvents; the distillation residue was stirred and cooled to room temperature; a pre-cooled solution of 58 g of sodium hydroxide (1.45 mol) in 190 ml of water was added dropwise to adjust the mixture to strong alkalinity; the mixture was allowed to stand for layer separation; the aqueous layer was extracted with 30 mL of dichloromethane each time for 3 times and then transferred to an environment-friendly treatment system for disposal; the oil layer and the dichloromethane extract were combined and heated under reflux, and the water was separated using a water separator; after the dichloromethane was removed by fractional distillation under atmospheric pressure, a total of 34.53 g of fraction was collected by fractional distillation under reduced pressure at 94-95° C./4.8 kPa, and a content of 99.5% was detected by gas chromatography (as shown in FIG. 1). The analysis results are shown in Table 3. It was confirmed by nuclear magnetic resonance spectroscopy (as shown in FIG. 2) that the target product was benzylamine with a molar yield of 80.2% (based on benzyl chloride).

TABLE 3

| Peak Height | Retention Time | Peak Area | | Content |
|---|---|---|---|---|
| [uV] | [min] | [uV*s] | Area % | [%] |
| 1003457 | 2.477 | 2991423 | 99.46857 | 99.46857 |
| 7613 | 2.616 | 15982 | 0.53143 | 0.53143 |
| Total: 1011070 | | 3007406 | 100.00000 | 100.00000 |

### Embodiment 2: Synthesis of 2-Chlorobenzylamine from 2-Chlorobenzyl Chloride

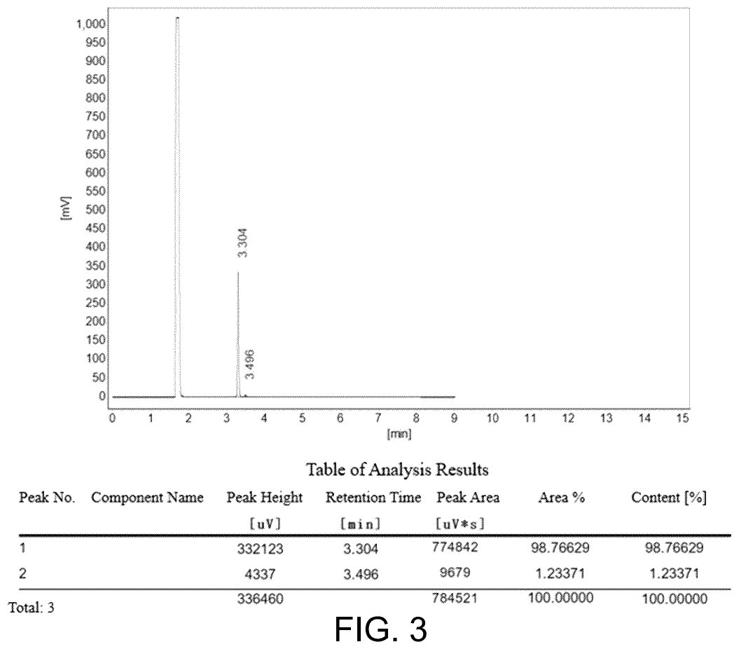

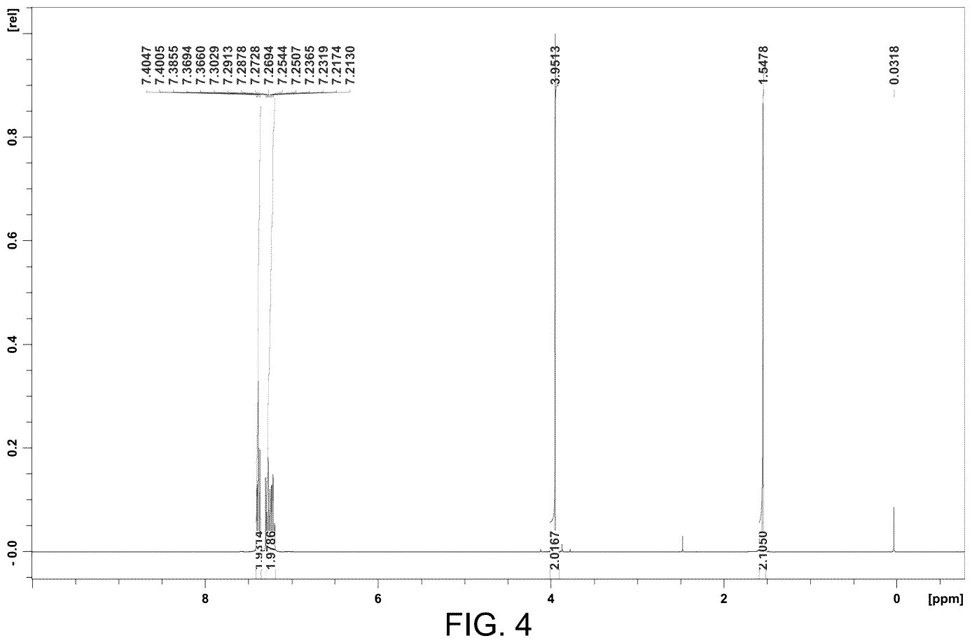

24.32 g of paraformaldehyde (0.81 mol) and 300 mL of methanol were added into a 500 mL four-necked flask equipped with a stirrer, a thermometer, a reflux condenser, and a dropping funnel to form a mixture; the mixture was cooled in an ice-water bath, and 74.51 g of aqueous ammonia with a mass concentration of 19.22% (0.84 mol) were added under stirring, with the temperature controlled below 30° C.; after the mixture was completely dissolved and clarified, stirring was continued for 20 min; then, 48.3 g of 2-chlorobenzyl chloride (0.3 mol) were added; the water bath temperature was raised to control the reaction temperature at 58-60° C.; after stirring for 6 h, the reaction was stopped and cooled to room temperature with cold water; 71.4 mL of 31.0% hydrochloric acid (0.7 mol) were added; the mixture was heated in a water bath, with the reaction temperature controlled at 40-45° C.; after reaction for 2 h, an atmospheric distillation apparatus was used to distill out all organic solvents; heating was stopped, and the distillation residue was cooled to room temperature; a pre-cooled solution of 48.0 g of sodium hydroxide (1.2 mol) in 72 mL of water was added dropwise under stirring to adjust the mixture to strong alkalinity; the mixture was allowed to stand for layer separation; the aqueous layer was extracted with 30 mL of benzene each time for 3 times and then transferred to an environment-friendly treatment system for disposal; the oil layer and the benzene extract were combined and heated under reflux, and the water was separated using a water separator; after the benzene was removed by fractional distillation under atmospheric pressure, a total of 34.63 g of fraction was collected by fractional distillation under reduced pressure at 84-86° C./0.9 kPa, and a content of 98.8% was detected by gas chromatography (as shown in FIG. 3). The analysis results are shown in Table 4. It was confirmed by nuclear magnetic resonance spectroscopy (as shown in FIG. 4) that the target product was 2-chlorobenzylamine with a molar yield of 80.5% (based on 2-chlorobenzyl chloride).

TABLE 4

| | | | | | | |
|---|---|---|---|---|---|---|
| Peak | Component | Peak Height | Retention Time | Peak Area | | Content |
| No. | Name | [uV] | [min] | [uV · s] | Area % | [%] |

| | | | | | |
|---|---|---|---|---|---|
| 1 | 332123 | 3.304 | 774842 | 98.76629 | 98.76629 |
| 2 | 4337 | 3.496 | 9679 | 1.23371 | 1.23371 |
| Total: 3 | 336460 | | 784521 | 100.00000 | 100.00000 |

### Embodiment 3: Synthesis of 4-Fluorobenzylamine from 4-Fluorobenzyl Chloride

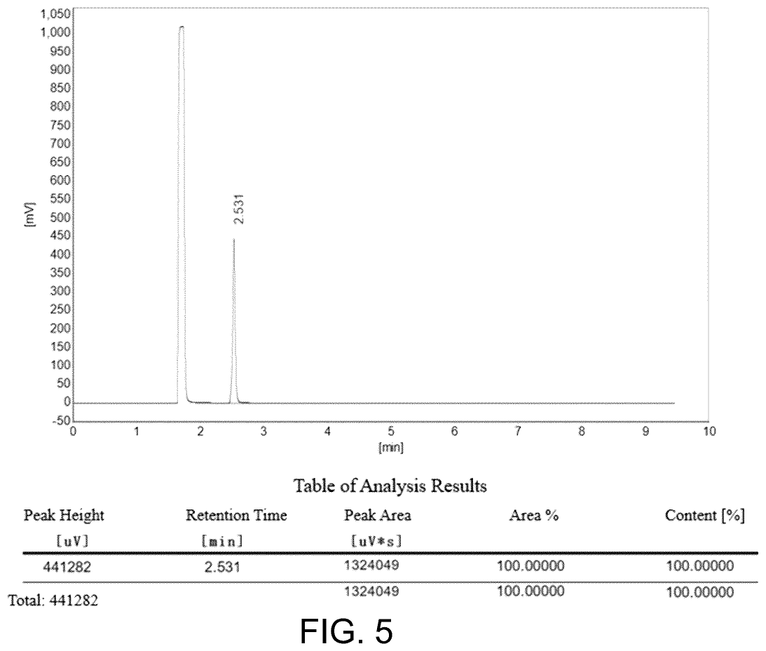

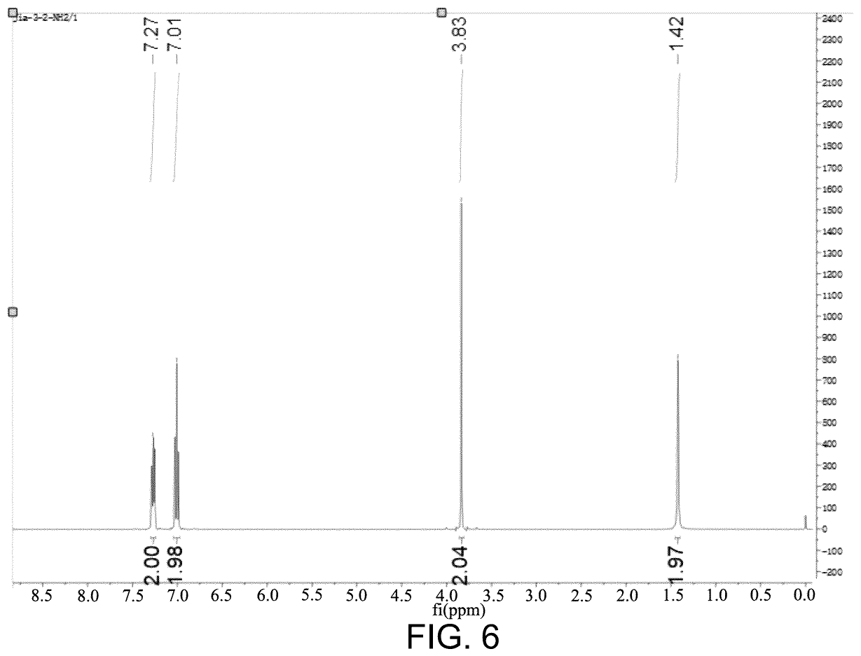

38.44 g of paraformaldehyde (1.28 mol) and 400 mL of methanol were added into a 1,000 mL four-necked flask equipped with a stirrer, a thermometer, a reflux condenser, and a dropping funnel to form a mixture; the mixture was cooled in an ice-water bath, and 88.3 g of aqueous ammonia with a mass concentration of 27.0% (1.4 mol) were added under stirring, with the temperature controlled below 30° C.; after the mixture was completely dissolved and clarified, stirring was continued for 30 min; then, 57.83 g of 4-fluorobenzyl chloride (0.4 mol) were added; the water bath temperature was raised to control the reaction temperature at 55-57° C.; after stirring for 5 h, the reaction was stopped and cooled to room temperature with cold water; 120.0 g of 36.5% hydrochloric acid (1.2 mol) were added; the mixture was heated in a water bath, with the reaction temperature controlled at 40-45° C.; after reaction for 4 h, an atmospheric distillation apparatus was used to distill out all organic solvents; the distillation residue was stirred for crystallization, cooled to 4° C. with ice water, and vacuum filtered until dry; the crystals obtained from the filtration were transferred to a 500 mL three-necked flask, 100 ml of water was added, and then a pre-cooled solution of 76.8 g of sodium hydroxide (1.92 mol) in 200 mL of water was added dropwise under stirring to adjust the mixture to strong alkalinity; the mixture was allowed to stand for layer separation; the aqueous layer was extracted with 30 mL of dichloromethane each time for 3 times and then transferred to an environment-friendly treatment system for disposal; the oil layer and the dichloromethane extract were combined and heated under reflux, and the water was separated using a water separator; after the dichloromethane was removed by fractional distillation under atmospheric pressure, a total of 41.53 g of fraction was collected by fractional distillation under reduced pressure at 63-64° C./1.0 kPa, and a content of 100% was detected by gas chromatography (as shown in FIG. 5). The analysis results are shown in Table 5. It was confirmed by nuclear magnetic resonance spectroscopy (as shown in FIG. 6) that the target product was 4-fluorobenzylamine with a molar yield of 83.0% (based on 4-fluorobenzyl chloride).

TABLE 5

| Peak Height | Retention Time | Peak Area | | Content |
|---|---|---|---|---|
| [uV] | [min] | [uV*s] | Area % | [%] |
| 441282 | 2.531 | 1324049 | 100.00000 | 100.00000 |
| Total: 441282 | | 1324049 | 100.00000 | 100.00000 |

### Embodiment 4: Synthesis of 4-tert-Butylbenzylamine from 4-tert-Butylbenzyl Chloride

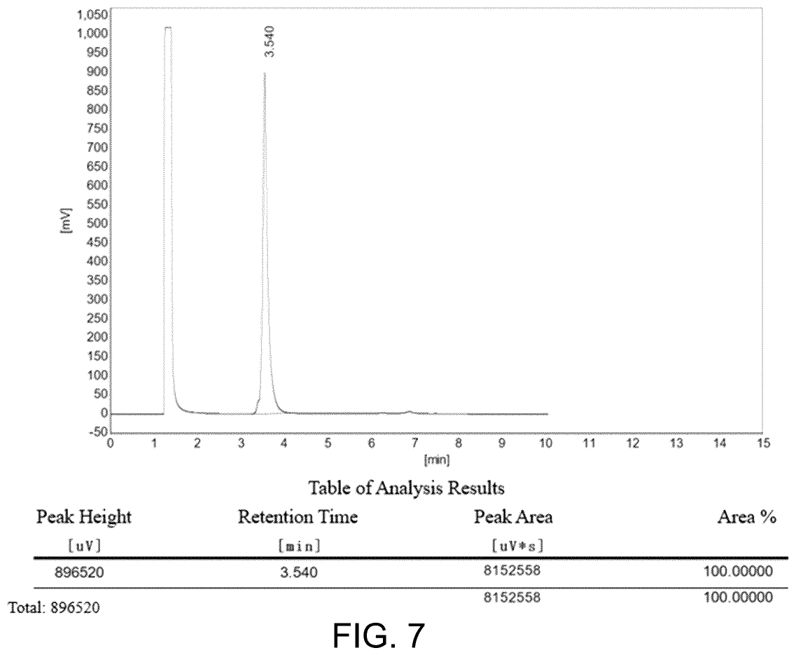

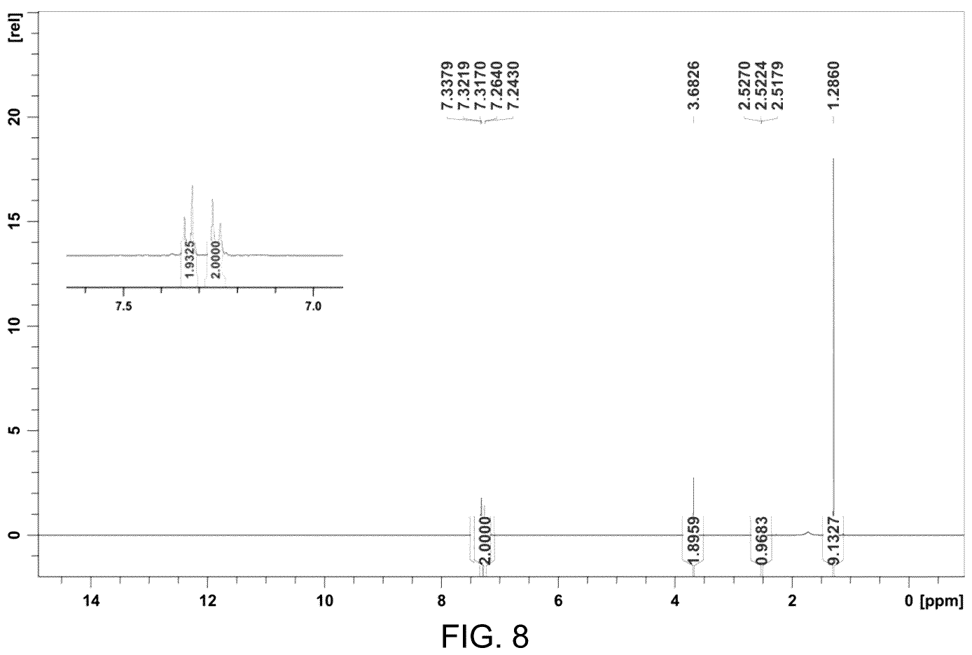

33.63 g of paraformaldehyde (1.12 mol) and 700 mL of methanol were added into a 1,000 mL four-necked flask equipped with a stirrer, a thermometer, a reflux condenser, and a dropping funnel to form a mixture; the mixture was cooled in an ice-water bath, and 88.85 g of aqueous ammonia with a mass concentration of 22.1% (1.153 mol) were added under stirring, with the temperature controlled below 30° C.; after the mixture was completely dissolved and clarified, stirring was continued for 30 min; then, 63.94 g of 4-tert-butylbenzyl chloride (0.35 mol) were added; the water bath temperature was raised to control the reaction temperature at 59-61° C.; after stirring for 7 h, the reaction was stopped and cooled to room temperature with cold water; 74.9 mL of 36.5% hydrochloric acid (0.8857 mol) were added; the mixture was heated in a water bath, with the reaction temperature controlled at 39-41° C.; after reaction for 3 h, an atmospheric distillation apparatus was used to distill out organic solvents while 200 mL of water was added dropwise until all the organic solvents were distilled out; heating was stopped, and the distillation residue was cooled to room temperature first and then further cooled to below 5° C. with ice water; the mixture was stirred for crystallization for 3 h and vacuum filtered until dry; the filter cake was transferred to a 500 mL three-necked flask, and a pre-cooled solution of 54.4 g of sodium hydroxide (1.36 mol) in 200 mL of water was added dropwise under stirring to adjust the mixture to strong alkalinity; the mixture was allowed to stand for layer separation; the aqueous layer was extracted with 50 mL of methyl tert-butyl ether each time for 3 times and then transferred to an environment-friendly treatment system for disposal; the oil layer and the methyl tert-butyl ether extract were combined and heated under reflux, and the water was separated using a water separator; after the methyl tert-butyl ether was removed by fractional distillation under atmospheric pressure, a total of 47.19 g of fraction was collected by fractional distillation under reduced pressure at 92-94° C./0.5 kPa, and a content of 100.0% was detected by gas chromatography (as shown in FIG. 7). The analysis results are shown in Table 6. It was confirmed by nuclear magnetic resonance spectroscopy (as shown in FIG. 8) that the target product was 4-tert-butylbenzylamine with a molar yield of 82.6% (based on 4-tert-butylbenzyl chloride).

TABLE 6

| Peak Height | Retention Time | Peak Area | |
|---|---|---|---|
| [uV] | [min] | [uV*s] | Area % |
| 896520 | 3.540 | 8152558 | 100.00000 |
| Total: 896520 | | 8152558 | 100.00000 |

### Embodiment 5: Synthesis of 2,4-Dichlorobenzylamine from 2,4-Dichlorobenzyl Chloride

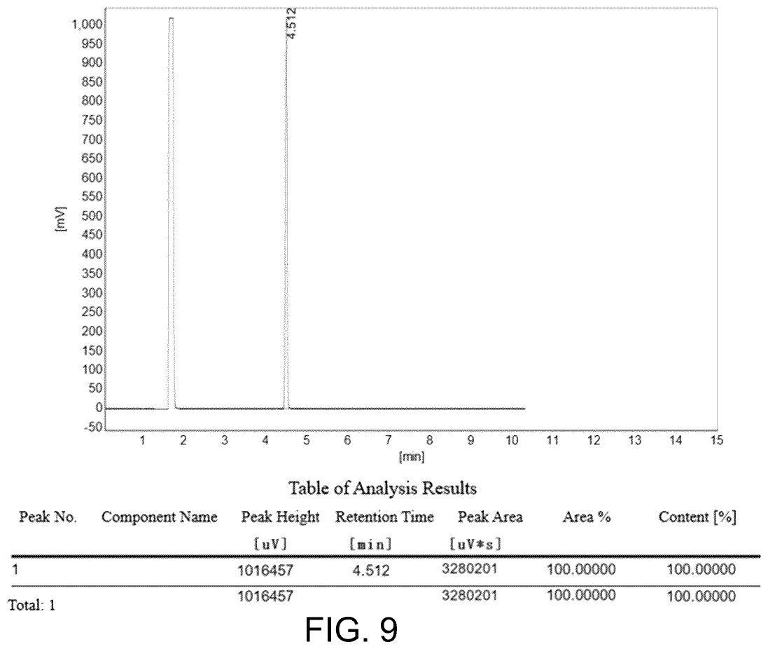

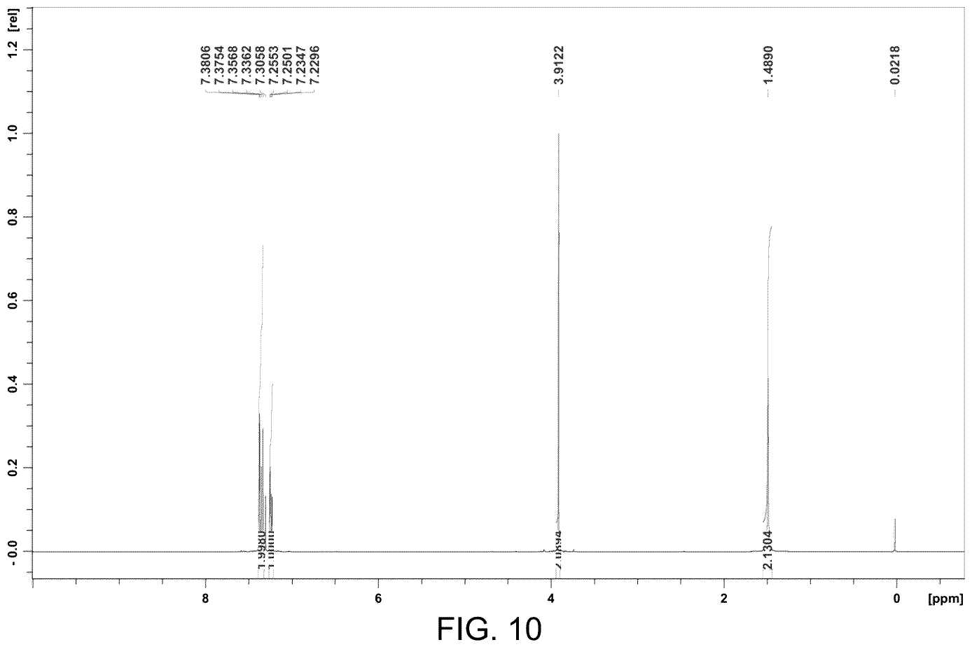

27.03 g of paraformaldehyde (0.9 mol) and 450 mL of methanol were added into a 1,000 mL four-necked flask equipped with a stirrer, a thermometer, a reflux condenser, and a dropping funnel to form a mixture; the mixture was cooled in an ice-water bath, and 74.11 g of aqueous ammonia with a mass concentration of 21.74% (0.9461 mol) were added under stirring, with the temperature controlled below 30° C.; after the mixture was completely dissolved and clarified, stirring was continued for 60 min; then, 58.64 g of 2,4-dichlorobenzyl chloride (0.3 mol) were added; the water bath temperature was raised to control the reaction temperature at 59-61° C.; after stirring for 4 h, the reaction was stopped and cooled to room temperature with cold water; 78.05 g of 36.5% hydrochloric acid (0.7805 mol) and 50 mL of water were added; the mixture was heated in a water bath, with the reaction temperature controlled at 40-45° C.; after reaction for 4 h, an atmospheric distillation apparatus was used to distill out organic solvents while 300 mL of water was added dropwise until all the organic solvents were distilled out; heating was stopped, and the distillation residue was cooled to room temperature; a pre-cooled solution of 48.0 g of sodium hydroxide (1.2 mol) in 100 mL of water was added dropwise under stirring to adjust the mixture to strong alkalinity; the mixture was allowed to stand for layer separation; the aqueous layer was extracted with 30 mL of dichloromethane each time for 3 times and then transferred to an environment-friendly treatment system for disposal; the oil layer and the dichloromethane extract were combined and heated under reflux, and the water was separated using a water separator; after the dichloromethane was removed by fractional distillation under atmospheric pressure, a total of 45.77 g of fraction was collected by fractional distillation under reduced pressure at 115-117° C./1.2 kPa, and a content of 100% was detected by gas chromatography (as shown in FIG. 9). The analysis results are shown in Table 7. It was confirmed by nuclear magnetic resonance spectroscopy (as shown in FIG. 10) that the target product was 2,4-dichlorobenzylamine with a molar yield of 86.7% (based on 2,4-dichlorobenzyl chloride).

TABLE 7

| | | | | | | |
|---|---|---|---|---|---|---|
| Peak | Component | Peak Height | Retention Time | Peak Area | | Content |
| No. | Name | [uV] | [min] | [uV · s] | Area % | [%] |

| | | | | | |
|---|---|---|---|---|---|
| 1 | 1016457 | 4.512 | 3260201 | 100.00000 | 100.00000 |
| Total: 1 | 1016457 | | 3260201 | 100.00000 | 100.00000 |

### Embodiment 6: Synthesis of 4-Bromobenzylamine from 4-Bromobenzyl Bromide

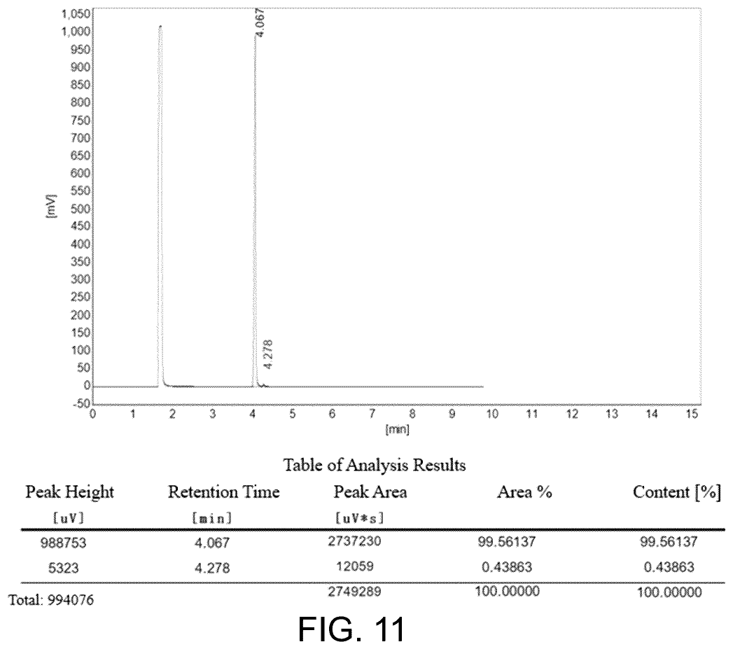

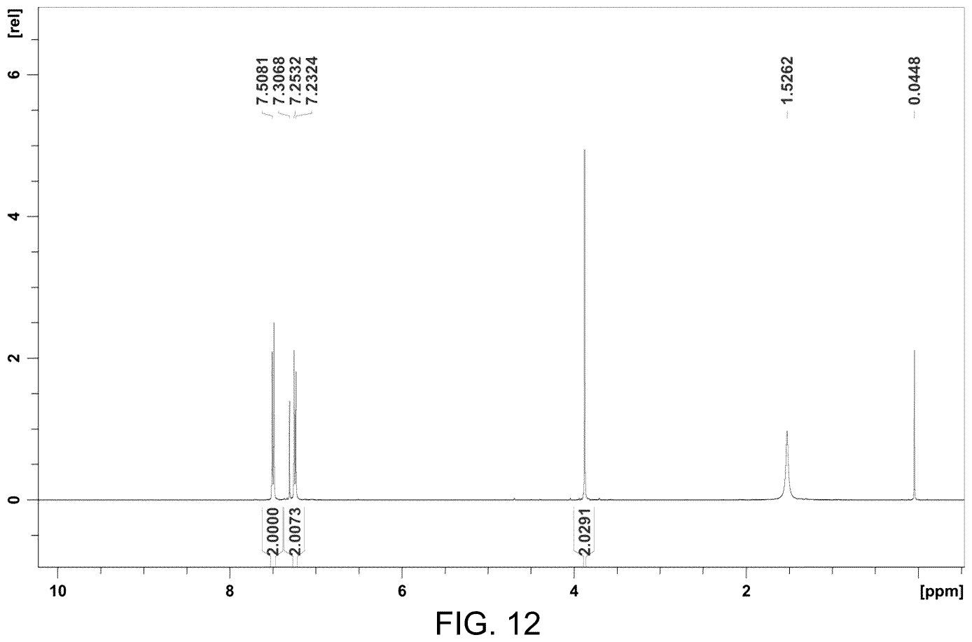

25.23 g of paraformaldehyde (0.84 mol) and 600 mL of methanol were added into a 1,000 mL four-necked flask equipped with a stirrer, a thermometer, a reflux condenser, and a dropping funnel to form a mixture; the mixture was cooled in an ice-water bath, and 63.69 g of aqueous ammonia with a mass concentration of 24.12% (0.9021 mol) were added under stirring, with the temperature controlled below 30° C.; after the mixture was completely dissolved and clarified, stirring was continued for 30 min; then, 74.98 g of 4-bromobenzyl bromide (0.3 mol) were added; the water bath temperature was raised to control the reaction temperature at 54-56° C.; after stirring for 8 h, the reaction was stopped and cooled to room temperature with cold water; 69.29 g of 36.5% hydrochloric acid (0.6929 mol) were added; the mixture was heated in a water bath, with the reaction temperature controlled at 44-46° C.; after reaction for 2 h, an atmospheric distillation apparatus was used to distill out organic solvents while 100 mL of water was added dropwise until all the organic solvents were distilled out; heating was stopped, and the distillation residue was cooled to room temperature; a pre-cooled solution of 44.0 g of sodium hydroxide (1.1 mol) in 100 mL of water was added dropwise under stirring to adjust the mixture to strong alkalinity; steam distillation was performed until the distillate was no longer cloudy; the distilled aqueous liquid was allowed to stand for layer separation; the aqueous layer was extracted with 40 mL of dichloromethane each time for 3 times and then transferred to an environment-friendly treatment system for disposal; the oil layer and the dichloromethane extract were combined and heated under reflux, and the water was separated using a water separator; after the dichloromethane was removed by fractional distillation under atmospheric pressure, a total of 48.17 g of fraction was collected by fractional distillation under reduced pressure at 100-102° C./0.5 kPa, and a content of 99.6% was detected by gas chromatography (as shown in FIG. 11). The analysis results are shown in Table 8. It was confirmed by nuclear magnetic resonance spectroscopy (as shown in FIG. 12) that the target product was 4-bromobenzylamine with a molar yield of 86.0% (based on 4-bromobenzyl bromide).

TABLE 8

| Peak Height | Retention Time | Peak Area | | Content |
|---|---|---|---|---|
| [uV] | [min] | [uV*s] | Area % | [%] |
| 988753 | 4.067 | 2737230 | 99.56137 | 99.56137 |
| 5323 | 4.278 | 12059 | 0.43863 | 0.43863 |
| Total: 994076 | | 2749289 | 100.00000 | 100.00000 |

### Embodiment 7: Synthesis of 1-Naphthylmethylamine from 1-Chloromethylnaphthalene

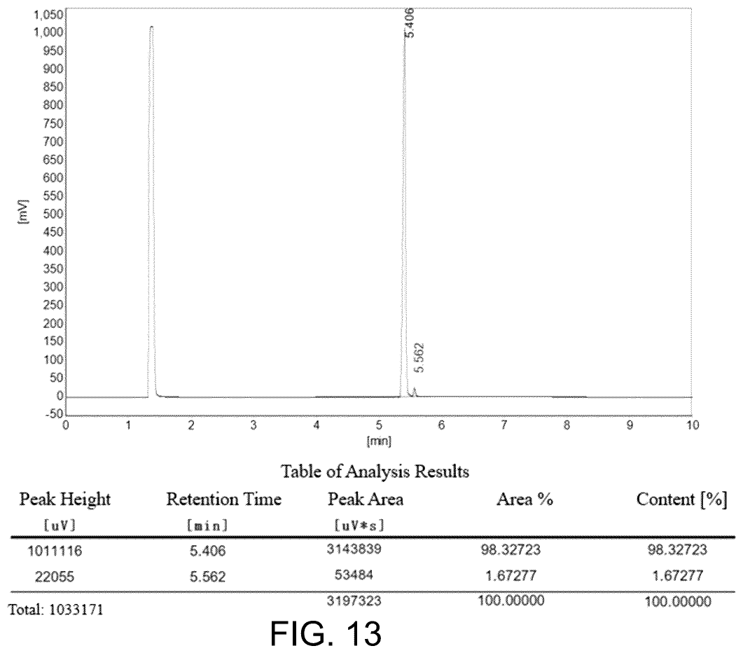

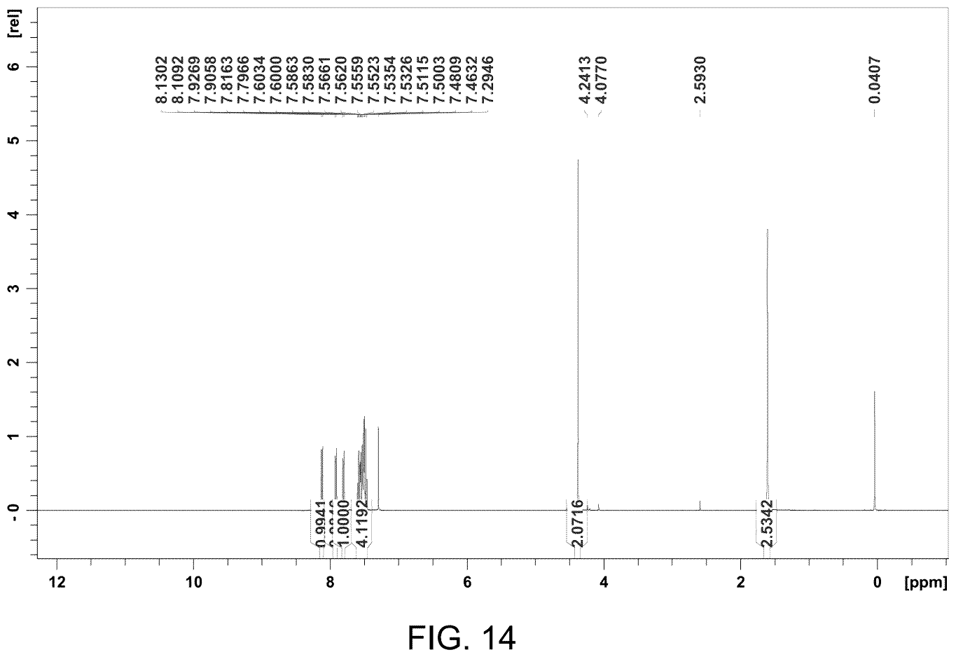

26.12 g of paraformaldehyde (0.87 mol) and 600 mL of methanol were added into a 1,000 mL four-necked flask equipped with a stirrer, a thermometer, a reflux condenser, and a dropping funnel to form a mixture; the mixture was cooled in an ice-water bath, and 65.33 g of aqueous ammonia with a mass concentration of 24.7% (approximately 0.9475 mol) were added under stirring, with the temperature controlled below 30° C.; after the mixture was completely dissolved and clarified, stirring was continued for 30 min; then, 52.99 g of 1-chloromethylnaphthalene (0.3 mol) were added; the water bath temperature was raised to control the reaction temperature at 56-58° C.; after stirring for 6.5 h, the reaction was stopped and cooled to room temperature with cold water; 200 mL of water and 78.96 g of 36.5% hydrochloric acid (0.7896 mol) were added; the mixture was heated in a water bath, with the reaction temperature controlled at 40-42° C.; after reaction for 3.5 h, an atmospheric distillation apparatus was used to distill out organic solvents while 300 mL of water was added dropwise until all the organic solvents were distilled out; heating was stopped, and the distillation residue was cooled to 50° C.; a pre-cooled solution of 46.74 g of sodium hydroxide (1.169 mol) in 100 mL of water was added dropwise under stirring to adjust the mixture to strong alkalinity; the mixture was allowed to stand for layer separation; the aqueous layer was extracted with 30 mL of dichloromethane each time for 3 times and then transferred to an environment-friendly treatment system for disposal; the oil layer and the dichloromethane extract were combined and heated under reflux, and the water was separated using a water separator; after the dichloromethane was removed by fractional distillation under atmospheric pressure, a total of 39.82 g of fraction was collected by fractional distillation under reduced pressure at 154-157° C./1.5 kPa, and a content of 98.3% was detected by gas chromatography (as shown in FIG. 13). The analysis results are shown in Table 9. It was confirmed by nuclear magnetic resonance spectroscopy (as shown in FIG. 14) that the target product was 1-naphthylmethylamine with a molar yield of 83.0% (based on 1-chloromethylnaphthalene).

TABLE 9

| Peak Height | Retention Time | Peak Area | | Content |
|---|---|---|---|---|
| [uV] | [min] | [uV*s] | Area % | [%] |
| 1011116 | 5.406 | 3143639 | 98.32723 | 98.32723 |
| 22055 | 5.562 | 53484 | 1.67277 | 1.67277 |
| Total: 1033171 | | 3197323 | 100.00000 | 100.00000 |

This disclosure still has many other embodiments, such as: synthesis of 2-methylbenzylamine from 2-methylbenzyl chloride, synthesis of 4-methylbenzylamine from 4-methylbenzyl chloride, synthesis of 4-chlorobenzylamine from 4-chlorobenzyl chloride, synthesis of 2-bromobenzylamine from 2-bromobenzyl bromide, synthesis of 2-fluorobenzylamine from 2-fluorobenzyl chloride, synthesis of 3,4-dichlorobenzylamine from 3,4-dichlorobenzyl chloride, synthesis of 2,4-difluorobenzylamine from 2,4-difluorobenzyl chloride, synthesis of 2,4-difluorobenzylamine from 2,4-difluorobenzyl bromide, synthesis of (trifluoromethyl)benzylamine from (trifluoromethyl)benzyl chloride, synthesis of benzhydrylamine from diphenylchloromethane, synthesis of 4-[2-(dimethylamino) ethoxy]benzylamine from 4-[2-(dimethylamino) ethoxy]benzyl chloride, synthesis of 4-[2-(dimethylamino) ethoxy]benzylamine from 4-[2-(dimethylamino) ethoxy]benzyl bromide, synthesis of 2-naphthalenemethylamine from 2-chloromethylnaphthalene, synthesis of 2-chloro-5-aminomethylthiazole from 2-chloro-5-chloromethylthiazole, synthesis of 2-chloro-5-aminomethylpyridine from 2-chloro-5-chloromethylpyridine, synthesis of pyridylmethylamine from chloromethylpyridine, synthesis of methoxybenzylamine from methoxybenzyl chloride, and synthesis of thiophenemethylamine from chloromethylthiophene. Due to space limitations, such embodiments will not be further elaborated herein. All syntheses of corresponding primary amines using halogenated compounds as raw materials according to the method of this disclosure or with only minor modifications thereto shall fall within the scope of protection of this disclosure.

What is claimed is:

1. A low-carbon and environment-friendly method for synthesizing a primary amine using a halogenated hydrocarbon as a raw material, wherein the method comprises the following steps:
1) placing formaldehyde or paraformaldehyde and a sufficient amount of an alcohol solvent into a four-necked flask equipped with a stirrer, a thermometer, a reflux condenser, and a dropping funnel, cooling in an ice-water bath and adding aqueous ammonia dropwise under stirring, controlling a temperature at a condition of 0° C. to a reflux temperature until a mixture is completely dissolved, and then continuing stirring for 0-2 h;
2) adding the halogenated hydrocarbon dropwise into the four-necked flask, stirring and heating in a water bath, and allowing for a full reaction at a condition of 20° C. to a reflux temperature for 0.5-24 h, wherein the halogenated hydrocarbon is: aryl methyl halides;
3) after the reaction is complete, cooling slightly, controlling the temperature at 0-60° C., stirring and adding 0.9 A-1.5 A equivalents of an inorganic acid or an organic acid dropwise, wherein A refers to an amount of a substance of ammonia contained in the aqueous ammonia minus an amount of a substance of the halogenated hydrocarbon, adding a small amount of water if necessary, and stirring and reacting at a temperature condition of 0-60° C. for 0.5-10 h; and
4) removing and replacing the reflux condenser with a distillation apparatus, adding an appropriate amount of water if necessary, stopping after all organic solvents are distilled out, and then proceeding with a post-treatment to obtain a finished primary amine product, wherein the primary amine is aryl methylamine,
wherein a molar feed ratio of each reactants being the halogenated hydrocarbon, the formaldehyde, and the ammonia contained in the aqueous ammonia is 1.0:(2.6-3.2):(2.6-3.5), and the amount of the substance of the ammonia contained in the aqueous ammonia is ≥an amount of a substance of the formaldehyde.

2. The low-carbon and environment-friendly method for synthesizing the primary amine using the halogenated hydrocarbon as the raw material of claim 1, wherein the molar feed ratio of each reactants being the halogenated hydrocarbon, the formaldehyde, and the ammonia contained in the aqueous ammonia is 3:8:8.

3. The low-carbon and environment-friendly method for synthesizing the primary amine using the halogenated hydrocarbon as the raw material of claim 1, wherein in the step 1), the temperature is controlled at preferably 15-30° C.; the alcohol solvent is selected from any one of methanol or ethanol; and a time for continuing stirring after dissolution is 20-60 min.

4. The low-carbon and environment-friendly method for synthesizing the primary amine using the halogenated hydrocarbon as the raw material of claim 1, wherein a feed amount of the alcohol is sufficient to ensure that the reaction mixture is in a solution state after the dropwise addition of the halogenated hydrocarbon in the subsequent step 2), and an actual amount of the alcohol varies depending on the halogenated hydrocarbon, and is greater than twice the amount of the substance of the formaldehyde.

5. The low-carbon and environment-friendly method for synthesizing the primary amine using the halogenated hydrocarbon as the raw material of claim 1, wherein in the step 2), after the dropwise addition of the halogenated hydrocarbon, a temperature for heating in the water bath is 30-65° C.; and a time for the full reaction is 1-12 h.

6. The low-carbon and environment-friendly method for synthesizing the primary amine using the halogenated hydrocarbon as the raw material of claim 1, wherein in the step 3), the temperature is appropriately controlled at 20-50° C.; a dropwise addition of the acid is 1.0 A-1.3 A equivalents of acid, wherein A refers to the amount of the substance of the ammonia contained in the aqueous ammonia minus the amount of the substance of the halogenated hydrocarbon; the acid is hydrochloric acid; and a time for stirring and reacting is 1-5 h.

7. The low-carbon and environment-friendly method for synthesizing the primary amine using the halogenated hydrocarbon as the raw material of claim 1, wherein in the step 4), the distillation apparatus employs atmospheric distillation or reduced-pressure distillation; the appropriate amount of water means that an amount of water added is sufficient to dissolve ammonium chloride precipitated during a later stage of the distillation, so that all the organic solvents are distilled out normally.

8. The low-carbon and environment-friendly method for synthesizing the primary amine using the halogenated hydrocarbon as the raw material of claim 1, wherein in the step 4), the post-treatment employs the following different methods, specifically:
method A: after slightly cooling a distillation residue, adjusting the distillation residue to strong alkalinity with an aqueous solution of 0.9 B-1.5 B equivalents of an alkali metal or alkaline earth metal hydroxide, standing for layer separation, extracting an aqueous layer with a suitable extractant and then transferring to an environment-friendly treatment system, combining an extract and an oil layer, removing water and performing fractional distillation under reduced pressure, and collecting an appropriate fraction to obtain the finished primary amine product; or
method B: cooling a distillation residue with ice water for crystallization, vacuum filtering until dry, adjusting obtained crystals to strong alkalinity with an aqueous solution of 0.9 B-1.5 B equivalents of an alkali metal or alkaline earth metal hydroxide, standing for layer separation, extracting an aqueous layer with a suitable extractant and then transferring to an environment-friendly treatment system, combining an extract and an oil layer, removing water and performing fractional distillation under reduced pressure, and collecting an appropriate fraction to obtain the finished primary amine product; or
method C: after adjusting a distillation residue to strong alkalinity by the method A or the method B, performing steam distillation, allowing a distillate to stand for layer separation, extracting an aqueous layer with a suitable extractant and then transferring to an environment-friendly treatment system, combining an extract and an oil layer, removing water and performing fractional distillation under reduced pressure, and collecting an appropriate fraction to obtain the finished primary amine product;
wherein in the above methods, B refers to an amount of a substance of the acid added plus the amount of the substance of the halogenated hydrocarbon; and the extractant is selected from an ether substance, a homologue of benzene, or partial halogenated hydrocarbons.

9. The low-carbon and environment-friendly method for synthesizing the primary amine using the halogenated hydrocarbon as the raw material of claim 8, wherein the aqueous solution of the alkali metal or alkaline earth metal hydroxide is an aqueous sodium hydroxide solution or an aqueous potassium hydroxide solution; a usage amount is 1.0 B-1.3 B, wherein B refers to the amount of the substance of the acid added plus the amount of the substance of the halogenated hydrocarbon;
the ether substance is selected from any one of diethyl ether, propyl ether, isopropyl ether, butyl ether, methyl tert-butyl ether, and anisole; the homologue of benzene is selected from any one of benzene, toluene, and xylene; the partial halogenated hydrocarbons are selected from dichloromethane; and
a method for removing the water is: removing water by using a method of drying with an inorganic salt following by filtration, wherein the inorganic salt is selected from any one of anhydrous potassium carbonate, anhydrous sodium carbonate, and anhydrous sodium sulfate; or, utilizing a characteristic of the extractant capable of forming an azeotropic mixture with water, heating under reflux and separating the water therein using a water separator.
